(19) United States
(12) Patent Application Publication
(10) Pub. No.: US 2024/0285149 A1
(43) Pub. Date: Aug. 29, 2024

(54) MEDICAL SYSTEM AND METHOD OF OPERATING THE SAME

(71) Applicant: OLYMPUS Winter & Ibe GmbH, Hamburg (DE)

(72) Inventor: Andreas MUECKNER, Schwarzenbek (DE)

(73) Assignee: OLYMPUS Winter & Ibe GmbH, Hamburg (DE)

(21) Appl. No.: 18/376,596

(22) Filed: Oct. 4, 2023

Related U.S. Application Data
(60) Provisional application No. 63/447,673, filed on Feb. 23, 2023.

(51) Int. Cl. A61B 1/00; A61B 5/00

(52) U.S. Cl. CPC A61B 1/00016; A61B 1/0002; A61B 5/7267

(57) ABSTRACT

A medical system, including: a surgical instrument having at least one sensor, the at least one sensor being configured to acquire measurement data, and a medical device configured to interact with the surgical instrument. Where the surgical instrument further includes: a first data communication interface; and a non-volatile storage medium for storing the acquired measurement data. The medical device further includes: a second data communication interface, wherein the first and the second data communication interface are configured to establish a data link and the surgical instrument is configured to communicate the measurement data stored in the non-volatile storage medium via the data link to the medical device, a processor comprising hardware, the processor being configured to generate output data from the measurement data; and an output interface for outputting of the output data.

### CROSS-REFERENCE TO RELATED APPLICATION

The present application is based upon and claims the benefit of priority from U.S. Provisional Application No. 63/447,673 filed on Feb. 23, 2023, the entire contents of which is incorporated herein by reference.

### BACKGROUND

### Field

The present disclosure relates to a medical system, and more particularly to a medical system comprising a surgical instrument and a medical device, which are configured to interact with each other. Furthermore, the present disclosure relates to a method of operating such a medical system.

### Prior Art

Surgical instruments, such as scalpels and forceps, and modern surgical instruments, such as electrosurgical instruments, are generally known. Surgical instruments can be handheld devices, such as a classical scalpel or electrosurgical forceps applied for occlusion of blood vessels for halting of bleeding in a process of electrocoagulation. In minimally invasive surgery, the surgical instruments are used in combination with an endoscope. An endoscope, which is not only applied for inspection and diagnosis but also for surgery, comprises one or more working channels projecting through the shaft of the endoscope from a hand piece at the proximal end to a tip at the distal end. Special surgical instruments can be inserted through the working channel for execution of surgical procedures in a surgical area located in front of the endoscope tip. Furthermore, many endoscopes comprise an imaging system, which guides light to the surgical area and captures images of the surgical field. For image capture, the endoscope comprises an imaging optic, which is integrated in the tip.

Endoscopes interact with medical devices such as camera heads that can be coupled to the hand piece at the proximal end, HF-generators for supplying the surgical instruments with a HF signal for execution of electrosurgical procedures, image processors performing image processing of image data, which is for example captured by the camera head, and the like.

### SUMMARY

Within the context of this specification, a surgical instrument shall be considered a tool, which is used during surgery in direct interaction and for manipulation of the body of a patient. An endoscope shall also be considered a surgical instrument although the direct surgical procedure is often performed by a specialized surgical tool that is inserted through a working channel of the endoscope. Further examples for surgical instruments are given above. A unit interacting with the surgical instrument within the context of use of the surgical instrument shall be considered a medical device. In contrast to the surgical instrument, the medical device is not in direct contact with the patient's body and is not configured for direct manipulation of the body of a patient.

A surgical instrument and a medical device shall be considered as interacting with each other if the two entities do functionally cooperate with each other within the context of use of the surgical instrument. For example, an endoscope interacts with a camera head and a camera processor during use, namely during imaging of e.g. a surgical field, which is located in front of a tip of the endoscope shaft. Furthermore, by way of an example, an electrosurgical electrode such as a wire loop or any other type of mono or bipolar electrode, which guided to the surgical field through the endoscope working channel, interacts with a HF-generator during use of the endoscope. The HF-generator provides the electrode with suitable HF-signals, which can be applied during an electrosurgical treatment, for example to assist or enhance coagulation. The interaction between the surgical instrument and the medical device directly takes place during use of the surgical instrument.

However, a surgical instrument and a medical device shall also be considered as interacting with each other if there is no direct and timely coincident cooperation but timely subsequent functional cooperation. For example, a handheld device or an endoscope has to be reprocessed after use. The cleaning and disinfection is typically performed by a reprocessing device, which is a cleaning and disinfecting device. The reprocessing device, within the context of this specification, is considered to interact with the surgical instrument. This is because appropriate reprocessing has to be performed for accurate and successful (repeated) use of the surgical instrument.

An object is to provide a medical system comprising a surgical instrument and a medical device, which are configured to interact with each other, wherein the medical system can be enhanced with respect to its functionality.

Such object can be solved by a medical system, comprising:
a surgical instrument having at least one sensor configured to acquire measurement data, and a medical device configured to interact with the surgical instrument, wherein
the surgical instrument further comprises a first data communication interface and a non-volatile storage medium for storing the acquired measurement data, and wherein
the medical device further comprises
   a second data communication interface, wherein the first and the second data communication interface are configured to establish a data link and the surgical instrument is configured to communicate measurement data stored in the non-volatile storage medium via the data link to the medical device,
   a processor comprising hardware, the processor being configured to generate output data from the measurement data, and
   an output interface for outputting of the output data.

Traditionally, there are a few surgical instruments, for example endoscopes, that are equipped with electronic sensors. In the course of digitalization, more and more sensors are integrated in surgical instruments to provide a so-called “smart instrument”. For example, the sensor can be a pressure sensor. This pressure sensor can be integrated in the surgical instrument. For example, the pressure sensor can be integrated in a tool or surgical instrument that can be used in combination with an endoscope. It can be configured to measure a contact pressure between a distal end or tip of the surgical instrument and a tissue of a body. The surgical instrument can be for example an electrode, which can be used during minimally invasive electrosurgery and can be applied during a surgical procedure to assist coagulation for halting of bleeding. The measurement values of the contact pressure can be used for assessing a coagulation process that can be initiated or supported with the surgical instrument. Furthermore, the pressure values can be used to analyze mechanical stress that can be applied on either the tissue or the surgical instrument during use.

The surgical instrument can acquire the measurement data and can store it on the non-volatile storage medium. It is therefore not necessary that the surgical instrument is in permanent data communication with a further device, capable of storing data, for example with a medical device such as a camera unit or a HF-generator.

Another example for a sensor can be a temperature sensor. For example, the temperature of the surgical instrument, furthermore by way of an example the temperatures at various different locations or points of the surgical instrument can be acquired and the measurement values can be stored in the non-volatile storage medium. For example, there can be a first pressure sensor that can be configured to acquire a measurement value, which can be a temperature of the surgical instrument used in combination with the endoscope. The temperature sensor can be for example configured to measure a temperature of the electrode used during electrosurgery. Furthermore, there can be a second sensor, which can be configured to detect a temperature of the endoscope shaft, for example near to the distal end thereof. This measurement value can be useful with respect to the question whether or not the shaft of the endoscope potentially suffers from overheating during use of the electrosurgical tool. According to another scenario, temperature data can be acquired during reprocessing of the surgical instrument. This temperature data can be used to analyze the reprocessing process. Quality control can be performed, efficiency and reliability of the process can be measured or at least estimated. For example, a time sequence of the temperature values can be analyzed with respect to the question whether certain parts of the endoscope or the surgical instrument are heated to temperatures higher than a predetermined threshold value, so as to ensure that the reprocessing process was performed with sufficient intensity and high enough temperatures.

Surgical instruments have limited installation space for electronic components. Hence, the resources that can be integrated in the surgical instrument are also limited. In other words, even in view of the fact that the computing power of small microprocessors will probably increase over time, there will be hardly enough computing power for performing sophisticated data analysis of the acquired measurement data directly in the surgical instrument. Even if the microprocessor can cope with the limited installation space, the next limit will be the available energy for operation of the processor. In view of this situation, the medical system can provide a solution in which the surgical instrument acquires data, temporarily stores it and communicates the data upon occasion. In other words, the data can be transmitted when the data link between the first and the second data communication interface can be established. The data, which can be captured by the surgical instrument can be then communicated to the medical device.

The medical device can be an edge computing device. The medical device can be configured to communicate with further devices, for example with further medical devices or with other devices, such as a server or computer in a medical cloud. The medical device can also be configured to communicate with further surgical instruments. In other words, the medical device can be configured to communicate with more than one surgical instrument.

The medical device can be an edge computing device within the common definition of edge computing. Typically, edge computing is a distributed computing paradigm that brings computation and data storage closer to the sources of data. The medical device can be configured to analyze the data, which can be captured by the surgical instrument. The results but not the raw data can be communicated to further devices, for example to a cloud. The edge computing concept can improve response times and save bandwidth in the network. In other words, the edge computing approach can release the network from extensive data load while still providing the option to forward results of the analysis via the network or cloud to further devices, for example to servers or other medical devices in a medical cloud.

The medical device, which operates as the edge computing device, can be for example a camera head, a camera processor, a reprocessing device (washer disinfector), a HF-generator or any other medical device, which can be configured to interact with the surgical instrument.

The medical device can have a grid connection for the power supply line. The medical device can also be battery powered.

The medical system can be configured in that the surgical instrument comprises an auxiliary processor, which can be configured to preprocess the measurement data, and wherein the surgical instrument can be configured to communicate preprocessed measurement data via the data link to the medical device and the processor of the medical device can be configured to generate the output data from the preprocessed measurement data.

Preprocessing of measurement data can be a separation of meaningful data from remaining data. As a result, the surgical instrument can submit only the meaningful data to the medical device, which acts as an edge device. In this case, the medical device can perform the deeper data analysis and can for example trigger further action. This measure can also help to reduce the needed bandwidth for the data connection. The filter process can be implemented in the auxiliary processor of the surgical instrument by way of a threshold filter or by any other type of a filter function. It can also be possible to perform the data filtering by help of an AI model that can be implemented in the auxiliary processor. Summarizing, the surgical instrument can be configured to work conventionally and it can apply a pre-filter function and submit only the data to medical device that pass the filter function. This can be performed by way of a continuous stream or based on an event driven concept. The medical device can perform the more powerful computing tasks. Furthermore, there can be real time features and offline features that can be derived from the sensor signals. Real time features can be processed for example by an AI and the results can be used to trigger an action. This processing can either be performed on the auxiliary processor of the surgical instrument or on the processor of the medical device. An example for such a real time feature can be the sensing of a critical movement and the corresponding trigger can be a warning. If the analysis of the sensor signals can be performed on the processor of the medical device, a data link between the surgical instrument and the medical device is a prerequisite.

Offline features can be sensor signals that are processed and evaluated. For example, there can be a critical acceleration incident, which can be for example due to an accident and potential damage of the surgical instrument. This data can be preprocessed by the auxiliary processor in the surgical instrument and the data and/or the analysis of the data set can be communicated to the medical device upon occasion, which means when the data connection between the two devices can be established.

For the real time features, even if an immediate action can be triggered, data samples of the event, which caused the trigger, can be transferred to the medical device. The medical device can be configured to combine the data with data from other surgical instruments or other medical devices or with data, which was received by the surgical instrument earlier. The data can be combined and subsequently used to perform a further, more in-depth, analysis to optimize a neural network. This neural network can form the basis of an AI which can be implemented in a medical device or can form the basis of an AI that can be implemented in a surgical instrument. The surgical instrument can comprise a pre-trained neural network, which can be a static neural network, which means that the parameters of the network are not changed or amended by the surgical instrument itself. The optimization of the neural network can be performed based on a final decision for example with respect to a damage of the surgical instrument or on any other basis.

In addition to this, a medical instrument can be configured to estimate missing data from a first surgical instrument by using data from other connected surgical instruments or even by data received from other medical devices. This data estimation, which can be performed to supplement data that is missing, can be executed by an AI that can be implemented in a medical device.

According to another aspect, the system can be configured in that the auxiliary processor can comprise an auxiliary input interface through which the measurement data can be provided as an input feature to an auxiliary artificial intelligence (AI) model, wherein the auxiliary processor can be configured to perform an inference operation, in which the measurement data can be applied to the auxiliary AI model, to generate the preprocessed measurement data.

The auxiliary artificial intelligence model can be based on a static pre-trained neural network. This can be advantageous in that the limited resources that can be implemented in the surgical instrument, for example due to limited installation space and limited available electrical energy can be used in the best possible way. The training of the AI that is underlying the auxiliary artificial intelligence model executed in the auxiliary processor can be performed in the medical device.

Furthermore, the medical system can be configured in that the processor can comprise an input interface through which the measurement data can be provided as an input feature to an artificial intelligence (AI) model, wherein the processor can be configured to perform an inference operation in which the measurement data can be applied to the AI model to generate the output data.

The data analysis using the AI model can be performed at the medical device. In this context, the medical device can operate as a typical edge computing device. The raw data or preprocessed data of the surgical instrument, which can essentially be the measurement data, can be processed by the AI model in the medical device.

The parameters of the AI model can be communicated, for example, to other medicals devices performing a similar task. The parameters of the AI model can be updated between the various medical devices through a data link provided by a medical cloud. In other words, parameters of the AI model can be communicated from a first medical device to a cloud computer in the medical cloud and this cloud computer can distribute updated parameters of the AI model to further second medical devices that are participating in the medical cloud. By way of this concept, the network of the medical cloud can be released from massive data load, which would occur if the AI model is run on a central server. This is because in a centralized concept, all surgical instruments can communicate the raw data via the network to the server, which runs the AI model. This, however, causes high data traffic in the network. With the edge computing approach, the network can be released from this high data load. Nevertheless, advantages, which can result from the numerous application and training of the AI model by various medical devices, can be exploited. The AI model can be self-trained or user-trained by the numerous medical devices, which communicate via the medical cloud and can share the parameters of the AI model. The extensive use of the AI model can enhance the parameters of the model. Because the parameters can be communicated back to the medical devices, every single medical device running the AI model can benefit from the enhancement of the parameters of the AI model.

According to still another aspect, the medical system can be configured in that the medical device can be configured to communicate the output data from the second data communication interface via the data link to the first data communication interface of the surgical instrument.

Furthermore, the processor can be configured to train the neural networking underlying the auxiliary artificial intelligence model with the measurement data. Furthermore, the processor can be configured to generate an updated set of parameters of the static neural network as output data.

When the results of the data analysis are communicated back to the surgical instrument, the “smartness” of the instrument can be enhanced. This also applies to the auxiliary artificial intelligence model, which can be run on the auxiliary processor of the surgical instrument. Furthermore, the surgical instrument can be enhanced in that for example the functionality of the surgical device and the performance of the surgical device can be enhanced. For example, an analysis of the contact pressure between a surgical tool and a tissue can be performed. The data can be temporarily stored on the surgical instrument and subsequently communicated to the medical device and analyzed by the same. For example, the AI model found some optimal range for the contact pressure in view of a successful coagulation result, that can be entered in the system by way of a user input. This information for training of the neural network underlying the auxiliary artificial intelligence model and a new parameter set can be sent back to the surgical instrument. Holding this information can enhance the surgical instrument with respect to its functionality. For example, it can enable the surgical instrument to analyze and to communicate the optimum contact pressure to a user. For example, the surgical instrument can comprise a display and can assist the user in finding the optimal contact pressure for achieving an optimum in coagulation by way of the display output. On a long term basis, this can enhance the functional scope and quality of the surgical instrument.

According to still another aspect, the medical system can be configured in that the second data communication interface of the medical device can be configured to establish a second data link to a cloud computer and wherein the medical device can be configured to communicate the output data via the second data link to the cloud computer. Aspects concerning the use of the medical cloud have been mentioned above and shall not be repeated.

Such object can further be solved by a method of operating a medical system, which comprises a surgical instrument having at least one sensor, a first data communication interface and a non-volatile storage medium, the medical system further comprising a medical device having a second data communication interface, a processor comprising hardware and an output interface, the method comprising: acquiring measurement data with at least one sensor and storing the acquired data on the non-volatile data storage medium of the surgical instrument, establishing a data link between the first and the second data communication interface, communicating measurement data from the surgical instrument to the medical device, processing of the measurement data with the processor to generate output data, and outputting the output data via the output interface.

Same or similar advantages which have been mentioned with respect to the medical system can apply to the method of operating the medical system in the same or a similar way and shall be therefore not repeated.

Furthermore, the method can be configured in that the surgical instrument can comprise an auxiliary processor, and wherein the method can further comprise: preprocessing the measurement data with the auxiliary processor, communicating the preprocessed measurement data via the data link from the surgical instrument to the medical device and generating the output data from the preprocessed measurement data with the processor of the medical device.

According to another aspect, the auxiliary processor can comprise an auxiliary input interface and an auxiliary artificial intelligence (AI) model, the method can further comprise: inputting the measurement data to the auxiliary input interface, providing the measurement data as an input feature to the auxiliary artificial intelligence (AI) model, performing an inference operation by the auxiliary processor by applying the measurement data to the auxiliary AI model, generating preprocessed measurement data as output data from an output of the auxiliary AI model.

In still another aspect, the method can be enhanced in that the auxiliary artificial intelligence model can be based on static pre-trained neural network.

Furthermore, the method can be further configured in that the processor can comprise an input interface and an artificial intelligence (AI) model, the method can comprise: inputting the measurement data to the input interface, providing the measurement data as an input feature to the artificial intelligence (AI) model, performing an inference operation by the processor by applying the measurement data to the AI model, generating output data from an output of the AI model.

In still another aspect, the method can be configured in that it can further comprise communicating the output data from the second data communication interface of the medical device via the data link to the first data communication interface of the surgical instrument.

Furthermore, the method can be configured in that the processor can be configured to train the neural network underlying the auxiliary artificial intelligence (AI) model with the measurement data and to generate an updated set of parameters of the static neural network as output data.

According to another aspect, the method can further comprise establishing a second data link between the second data communication interface of the medical device and a cloud computer, communicating the output data from the medical device via the second data link to the cloud computer.

### BRIEF DESCRIPTION OF THE DRAWINGS

Further characteristics will become apparent from the description of the embodiments together with the claims and the included drawings. Embodiments can fulfill individual characteristics or a combination of several characteristics.

The embodiments are described below, without restricting the general intent of the invention, based on the exemplary embodiments, wherein reference is made expressly to the drawings with regard to the disclosure of all details that are not explained in greater detail in the text. In the drawings:

FIG. 1 illustrates a simplified illustration of a medical system,

FIG. 2 illustrates a further simplified illustration of a medical system having medical devices that are linked to each other via a medical cloud,

FIG. 3 illustrates a flowchart of a method of operating the medical system,

FIG. 4 illustrates a schematic diagram of a medical system, in which the medical device runs an AI model, and

FIG. 5 illustrates another schematic diagram of a medical system, in which the surgical instrument runs an AI model.

In the drawings, the same or similar types of elements or respectively corresponding parts are provided with the same reference numbers in order to prevent the item from needing to be reintroduced.

### DETAILED DESCRIPTION

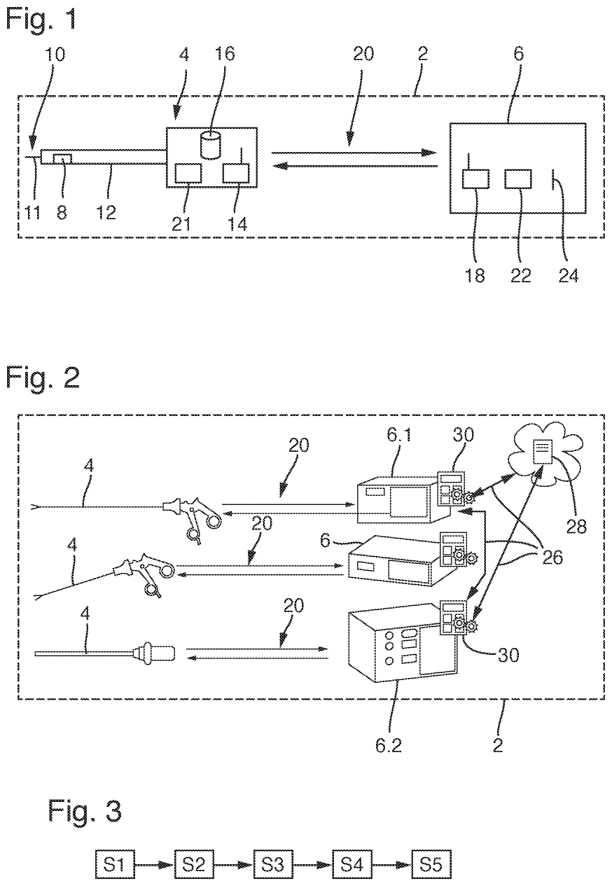

FIG. 1 is a simplified illustration of a medical system 2 comprising a surgical instrument 4 and a medical device 6. The surgical instrument 4 is for example an endoscope or a handheld surgical instrument. The medical device 6 is for example a camera processor, a reprocessing device or a HF-generator. The surgical instrument 4 and the medical device 6 are configured to interact with each other. The surgical instrument 4 and the medical device 6 interact with each other in that they functionally cooperate during use of the surgical instrument 4. For example, the endoscope interacts with the camera processor or the handheld surgical instrument interacts with the HF-generator during use. Image data, which is captured by the camera (not shown) of the endoscope, can be processed by the image processor. If the medical device 6 is for example a HF-generator, the medical device 6 and the handheld surgical instrument 4 cooperate in that the HF-generator provides the instrument 4 with a HF-signal to perform a surgical treatment. If the medical device 6 is a reprocessing device, the surgical instrument 4 and the medical device 6 interact in that the surgical instrument 4 has to be cleaned and disinfected using the reprocessing device after use.

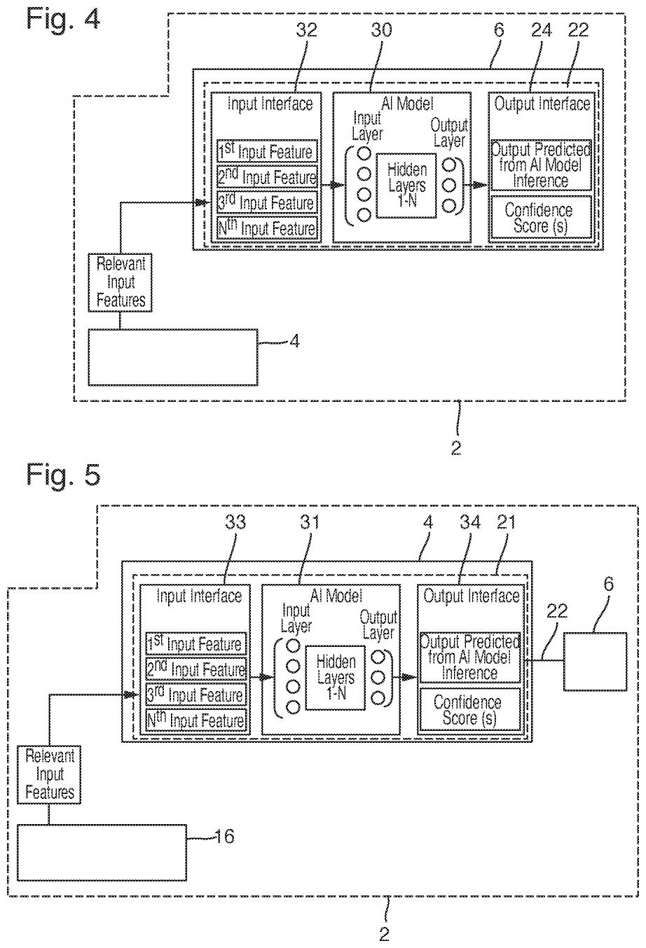

The surgical instrument 4 comprises at least one sensor 8, which is for example a pressure sensor or a temperature sensor. By way of an example only, the surgical instrument 4 in FIG. 1 comprises only one sensor 8. However, the surgical instrument 4 can comprise more than one sensor. The sensor 8 is for example configured to measure a contact pressure between a tip 10 of a specialized surgical tool 11 that can be guided through a working channel of the endoscope. The sensor 8 can be placed in a shaft 12 of the endoscope and a tissue during a surgical procedure. The surgical instrument 4 further comprises a first data communication interface 14 and a non-volatile storage medium 16 for storing acquired measurement data of the sensor 8. The medical device 6 comprises a second data communication interface 18. The first data communication interface 14 and the second data communication interface 18 are configured to establish a data link 20. The data link 20 can be a wired data link or a wireless data link. Furthermore, the data link 20 can be a bidirectional data link as it is illustrated in FIG. 1 (in which case each are transceivers). Is also possible that the data link 20 is a mono-directional data link, wherein only the surgical instrument 4 sends data to the medical device 6 (in which case one is a transmitter and the other a receiver). The surgical instrument 4 is configured to communicate measurement data, which is stored in the non-volatile storage medium 16 via the data link 20 to the medical device 6. The medical device 6 further comprises a processor 22 comprising hardware for generating output data from the measurement data. The output data is provided at an output interface 24 of the medical device 6. From this output interface 24, the output data can be sent back to the surgical instrument 4 or can be communicated to further devices as will be outlined further below. The processor 22 of the medical device 6 can be configured to execute an artificial intelligence model. This will be outlined with reference to FIG. 4 further below. Processor 22 can be configured as dedicated hardware circuits or a hardware processor executing a series of software instructions.

The data link 20 can be a wired data link, if the surgical instrument 4 is for example an endoscope and the medical device 6 is a video processor. In such a scenario, a high amount of data is communicated from the camera system of the endoscope to the image processor. This can be performed via a wired data link. If the surgical instrument 4 is for example a handheld surgical tool and the medical device 6 is a reprocessing device, the data link 20 can be a wireless data link. Use of the wireless data link permits data transmission to occur during reprocessing of the surgical instrument 4. The wireless data link can be established based on various technological standards such as WLAN, Bluetooth etc.

The surgical instrument 4 can comprise an auxiliary processor 21 comprising hardware, which is configured to preprocess measurement data, which is captured by the sensor 8. Processor 21 can be configured as dedicated hardware circuits or a hardware processor executing a series of software instructions. The surgical instrument 4 is further configured to communicate the preprocessed measurement data via the data link 20 to the medical device 6. In this case, the processor 22 of the medical device 6 generates or computes the output data, which is provided at the output interface 24, from the preprocessed measurement data.

In FIG. 2, there is a simplified illustration of a medical system 2, in which second data communication interface 18 (see FIG. 1) of the medical device 6 is configured to establish a second data link 26 to a cloud computer 28. The second data link 26 can be a wireless or a wired data link. The medical device 6 is configured to communicate the output data 30 via the second data link 26 to the cloud computer 28. The second data link 26 can be a bidirectional data link. For example, a first medical device 6.1 can communicate the output data 30 via the second data link 26 to the cloud computer 28. The cloud computer 28 can process the output data or can directly forward the output data to a second medical device 6.2 via the second data link 26. According to another example, the first medical device 6.1 and the second medical device 6.2 can establish a direct second data link, which is illustrated by a double arrow between these two devices. Data can be directly communicated between the two medical devices 6.1, 6.2 in the same or similar way as it was outlined for the data communication with the cloud computer 28. The output data 30 can be for example parameters of an AI model, which is executed by the medical devices 6.1, 6.2. The medical devices 6.1, 6.2 can share the optimized parameters of the AI model. The optimization of the parameters of the AI model can be performed either in the medical devices 6.1, 6.2 or in the cloud computer 28.

In the medical system 2, which is illustrated in FIG. 2, there is also a medical device 6, that does not operate a second data connection 26 to the cloud computer 28. Such a standalone medical device 6 does not participate in the medical cloud.

FIG. 3 illustrates a flowchart of a method of operating the medical system 2. In step S1, measurement data is acquired with the sensor 8 and stored on the non-volatile data storage medium 16 of the surgical instrument 4. In step S2, the data link 20 between the first and the second data communication interface 14, 18 is established. This is for example performed after use of the surgical instrument 4 in a surgical procedure, for example, when the surgical instrument 4 is put into a reprocessing device as the medical device 6. In step S3, the measurement data, which is stored in the non-volatile data storage medium 16, is communicated from the surgical instrument 4 to the medical device 6. In step S4, the measurement data is processed by the processor 22 of the medical device 6 to generate output data. For example, the measurement data is applied on the artificial intelligence model held by the processor 22. In step S5, the output data is output via the output interface 24. For example, the output data is communicated to further medical devices 6 via a cloud connection or the data is communicated back to the surgical device 4.

A data link 20 is not necessarily a one-to-one data connection between a particular surgical instrument 4 and the assigned medical device 6. The surgical instrument 4 can be configured to establish plurality of data links to various medical devices 6. Similarly, the medical device 6 can be configured to establish a plurality of different data links 20 to a plurality of surgical instruments 4.

FIG. 4 shows a schematic diagram of a medical system 2, in which the processor 22 of the medical device 6 implements an AI model 30. The AI model 30 is configured to output data, which is based on measurement data, which is acquired by the sensor 8 of the surgical instrument 4. The output data can be for example data related to an operating parameter of the surgical instrument 4.

In various embodiments, the medical device 6 includes an input interface 32 through which measurement data, which is specific to a certain surgical instrument 4, is provided as an input feature to the artificial intelligence (AI) model 30. The processor 22 performs an inference operation in which the measurement data is applied to the AI model 30 to generate the output data. The output data can be communicated to the user via a user interface (UI). As it was previously mentioned, the user interface can be a display, which is arranged on the surgical instrument 4. The UI can also be a display connected to the medical device 6 (not shown).

The input interface 32 receives data via the data link 20 and the second data communication interface 18. The sensor 8 generates at least some of the input features of the artificial intelligence model 30. If the surgical instrument 4 comprises more than one sensor 8, a respective one of the sensors 8 can provide an input feature for the input interface 32 of the artificial intelligence model 30. For example, the surgical instrument 4 comprises a pressure sensor and the temperature sensor. Hence, the measurement values of the pressure sensor and the temperature values acquired by the temperature sensor can be used as input features. Because these sensors are configured to acquire a series of measurement data over time, also this time series of measurement values can be used as an input feature.

For example, the input interface 32 may transmit the measurement values directly to the AI model 30 after a therapeutic and/or diagnostic medical procedure, when the data link 20 is established. Additionally, or alternatively, the input interface 32 may be a classical user interface that simplifies interaction between a user and the medical device 6. For example, the input interface 32 can be a user interface through which the user may manually enter further information relative to the surgical instrument 4 or relative to the use of the surgical instrument 4. For example, certain observations made during use of the surgical instrument 4 or observed malfunctions can be manually entered via the input interface 32.

Additionally, or alternatively, the input interface 32 can provide the medical device 6 with access to a medical cloud and a cloud computer 28 from which one or more input features may be extracted. These input features can be for example measurement values of other surgical instruments 4.

Based on one or more of the input features, the processor 22 performs an inference operation using the AI model 30 to generate the output data. The output data can be for example operating parameters of the surgical instrument 4. When the sensor 8 acquires for example a contact pressure between a tip 10 of a surgical tool 11 of an endoscope and a tissue of a body, this measurement value can be input into the AI model 30. The output data, which is provided via the output interface 24, can be an operating parameter of the surgical instrument 4. This operating parameter configures the surgical instrument 4 in that for example a display on the surgical instrument 4 indicates an optimum contact pressure with respect to successful coagulation. Hence, the surgical instrument 4 will assist the surgeon when performing a coagulation treatment. The evaluation of the coagulation process can be manually entered as further input feature via the input interface 32. It can be used for further training of the AI model 30.

In FIG. 5, there is a surgical instrument 4, which comprises an auxiliary processor 21. The auxiliary processor 21 implements an auxiliary AI model 31. Furthermore, the auxiliary processor 21 comprises an auxiliary input interface 33. Through the auxiliary input interface 33, the measurement data is provided to the auxiliary artificial intelligence model 31 as an input feature thereof. The auxiliary processor 21 is configured to perform an interference operation, in which the measurement data is applied to the auxiliary AI model 31 to generate preprocessed measurement data, which is output via an auxiliary output interface 34.

The measurement data representing relevant input features to the AI model 31 can be taken from the storage medium 16. Alternatively, the measurement values can be directly provided by the sensor 8. The preprocessed measurement data, which is provided at the auxiliary output interface 34, can be communicated from the surgical instrument 4 to the medical device 6. The preprocessed measurement data can represent relevant input features that the medical device 6 receives at its input interface 32. In other words, the preprocessed data is sent from the auxiliary output interface 34 of the surgical instrument 4 to the input interface 32 of the medical device 6. The auxiliary artificial intelligence model 31 can be a static pre-trained neural network.

Furthermore, the processor 22 of the medical device 6 can be configured to train the neural network underlying the auxiliary artificial intelligence model 31 with the measurement data, which can be received from the surgical instrument 4. In other words, the surgical instrument 4 can deliver measurement data and in parallel preprocessed measurement data to the medical device 6. The training of the auxiliary artificial intelligence model 31 and the neural network underlying the same, results in generation of an updated set of parameters of a static neural network as output data of the medical device. This output data can be communicated back to the surgical instrument 4 in an attempt to enhance the auxiliary artificial intelligence model 31.

For example, the input interface 32 delivers the measurement data to an input layer of the AI model 30 which propagates the input features through the AI model 30 to an output layer.

The AI model 30, 31 can be provided on a computer system with the ability to perform tasks, without explicitly being programmed, by making inferences based on patterns found in the analysis of data. The AI model 30, 31 explores the study and construction of algorithms (e.g. machine-learning algorithms) that may learn from existing data and make predictions about new data. Such algorithms operate by building an AI model 30, 31 from example training data in order to make data-driven predictions or decisions expressed as outputs or assessments.

The following explanations pertained both, the artificial intelligence model 30 and the auxiliary artificial intelligence model 31.

There are two common modes for machine learning (ML): supervised ML and unsupervised ML. Supervised ML uses prior knowledge (e.g., examples that correlate inputs to outputs or outcomes) to learn the relationships between the inputs and the outputs. The goal of supervised ML is to learn a function that, given some training data, best approximates the relationship between the training inputs and outputs so that the ML model can implement the same relationships when given inputs to generate the corresponding outputs. Unsupervised ML is the training of an ML algorithm using information that is neither classified nor labeled, and allowing the algorithm to act on that information without guidance. Unsupervised ML is useful in exploratory analysis because it can automatically identify structure in data.

Common tasks for supervised ML are classification problems and regression problems. Classification problems, also referred to as categorization problems, aim at classifying items into one of several category values (for example, is this object an apple or an orange?). Regression algorithms aim at quantifying some items (for example, by providing a score to the value of some input). Some examples of commonly used supervised-ML algorithms are Logistic Regression (LR), NaïveBayes, Random Forest (RF), neural networks (NN), deep neural networks (DNN), matrix factorization, and Support Vector Machines (SVM).

Some common tasks for unsupervised ML include clustering, representation learning, and density estimation. Some examples of commonly used unsupervised-ML algorithms are K-means clustering, principal component analysis, and autoencoders.

Another type of ML is federated learning (also known as collaborative learning) that trains an algorithm across multiple decentralized devices holding local data, without exchanging the data. This approach stands in contrast to traditional centralized machine-learning techniques where all the local datasets are uploaded to one server, as well as to more classical decentralized approaches which often assume that local data samples are identically distributed. Federated learning enables multiple actors to build a common, robust machine learning model without sharing data, thus allowing to address critical issues such as data privacy, data security, data access rights and access to heterogeneous data.

In some examples, the AI model 30, 31 may be trained continuously or periodically prior to performance of the inference operation by the processor 21, 22. Then, during the inference operation, the measurement data as specific input features provided to the AI model 30, 31 may be propagated from an input layer, through one or more hidden layers, and ultimately to an output layer that corresponds to the output data.

For example, a measurement value of the sensor 8, which can be a pressure value or a temperature value, is provided as input features to the AI model 30 and used to generate the output, which can be one or more parameter(s) of the surgical instrument 4.

During and/or subsequent to the inference operation, the output may be communicated to the surgical instrument 2 and/or to a user via a user interface (UI) and/or automatically causes the medical device 6 performing a desired action. For example, the medical device 6 will inform a clinician of the AI generated output, this can e.g., be a report of the suggested new parameters of the surgical instrument 4 and the corresponding AI generated confidence level.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCES

2 medical system
4 surgical instrument
6, 6.1, 6.2 medical device
8 sensor
10 tip
11 surgical tool
12 shaft
14 first data communication interface
16 storage medium
18 second data communication interface
20 data link
21 auxiliary processor
22 processor
24 output interface
26 second data link
28 cloud computer
30 AI model
31 auxiliary AI model
32 input interface
33 auxiliary input interface
34 auxiliary output interface

What is claimed is:

1. A medical system, comprising:
a surgical instrument having at least one sensor, the at least one sensor being configured to acquire measurement data, and
a medical device configured to interact with the surgical instrument,
wherein the surgical instrument further comprises:
a first data communication interface; and
a non-volatile storage medium for storing the acquired measurement data,
the medical device further comprises:
a second data communication interface, wherein the first and the second data communication interface are configured to establish a data link and the surgical instrument is configured to communicate the measurement data stored in the non-volatile storage medium via the data link to the medical device,
a processor comprising hardware, the processor being configured to generate output data from the measurement data; and
an output interface for outputting of the output data.

2. The medical system according to claim 1, wherein the processor comprises an input interface through which the measurement data is provided as an input feature to an artificial intelligence (AI) model, wherein the processor is configured to perform an inference operation in which the measurement data is applied to the AI model to generate the output data.

3. The medical system according to claim 1, wherein the surgical instrument comprises an auxiliary processor comprising hardware, is the auxiliary processor being configured to preprocess the measurement data, and wherein the surgical instrument is configured to communicate preprocessed measurement data via the data link to the medical device and the processor of the medical device is configured to generate the output data from the preprocessed measurement data.

4. The medical system according to claim 3, wherein the auxiliary processor comprises an auxiliary input interface through which the measurement data is provided as an input feature to an auxiliary artificial intelligence (AI) model, wherein the auxiliary processor is configured to perform an inference operation, in which the measurement data is applied to the auxiliary AI model, to generate the preprocessed measurement data.

5. The medical system according to claim 4, wherein the auxiliary artificial intelligence (AI) model is based on a static pre-trained neural network.

6. The medical system according to claim 1, wherein the medical device is configured to communicate the output data from the output interface via the second data communication interface and the data link to the first data communication interface of the surgical instrument.

7. The medical system according to claim 5, wherein the processor is configured to train the neural network underlying the auxiliary artificial intelligence (AI) model with the measurement data and to generate an updated set of parameters of the static neural network as output data.

8. The medical system according to claim 1, wherein the second data communication interface of the medical device is configured to establish a second data link to a cloud computer and wherein the medical device is configured to communicate the output data via the second data link to the cloud computer.

9. A method of operating a medical system, which comprises a surgical instrument having at least one sensor, a first data communication interface and a non-volatile storage medium, the medical system further comprising a medical device having a second data communication interface, a processor comprising hardware and an output interface, the method comprising:
acquiring measurement data with the at least one sensor and storing the acquired data on the non-volatile data storage medium of the surgical instrument,
establishing a data link between the first and the second data communication interface,
communicating measurement data from the surgical instrument to the medical device,
processing of the measurement data with the processor to generate output data, and
outputting the output data via the output interface.

10. The method of operating the medical system according to claim 9, wherein the processor comprises an input interface and an artificial intelligence (AI) model, the method comprising:
inputting the measurement data to the input interface,
providing the measurement data as an input feature to the artificial intelligence (AI) model,
performing an inference operation by the processor by applying the measurement data to the AI model, and
generating output data from an output of the AI model.

11. The method of operating the medical system according to claim 9, wherein the surgical instrument comprises an auxiliary processor comprising hardware, and wherein the method further comprises:
preprocessing the measurement data with the auxiliary processor,
communicating the preprocessed measurement data via the data link from the surgical instrument to the medical device, and
generating the output data from the preprocessed measurement data with the processor of the medical device.

12. The method of operating the medical system according to claim 11, wherein the auxiliary processor comprises an auxiliary input interface and an auxiliary artificial intelligence (AI) model, the method further comprising:
inputting the measurement data to the auxiliary input interface,
providing the measurement data as an input feature to the auxiliary artificial intelligence (AI) model,
performing an inference operation by the auxiliary processor by applying the measurement data to the auxiliary AI model, and
generating preprocessed measurement data as output data from an output of the auxiliary AI model.

13. The method of operating a medical system according to claim 12, wherein the auxiliary artificial intelligence (AI) model is based on a static pre-trained neural network.

14. The method of operating the medical system according to claim 9, further comprising communicating the output data from the output interface via the second data communication interface of the medical device and via the data link to the first data communication interface of the surgical instrument.

15. The method of operating a medical system according to claim 13, wherein the processor is configured to train the neural network underlying the auxiliary artificial intelligence (AI) model with the measurement data and to generate an updated set of parameters of the static neural network as output data.

16. The method of operating the medical system according to claim 9, further comprising:
establishing a second data link between the second data communication interface of the medical device and a cloud computer,
communicating the output data from the medical device via the second data link to the cloud computer.
